(19) United States
(12) Patent Application Publication
(10) Pub. No.: US 2025/0179406 A1
(43) Pub. Date: Jun. 5, 2025

(54) METHOD OF OPERATING A FERMENTATION DEVICE

(71) Applicant: STRABAG Umwelttechnik GmbH, Dresden (DE)

(72) Inventors: Torsten Baumann, Dresden (DE); Martin Hummitzsch, Niederfrohna (DE); Thomas Büchner, Kreischa (DE)

(21) Appl. No.: 19/049,852

(22) Filed: Feb. 10, 2025

Related U.S. Application Data
(63) Continuation of application No. PCT/EP2022/072479, filed on Aug. 10, 2022.

(51) Int. Cl. C12M 1/06; C12M 1/00

(52) U.S. Cl. CPC C12M 27/06; C12M 29/18; C12M 29/20

(57) ABSTRACT

A method of operating a fermentation device has an elongated container and multiple stirrer devices. The method includes introducing substrate containing organic material via the at least one introduction opening, moving and mixing the substrate in the vessel via the rotationally driven stirrer devices, removing treated material via the at least one discharge opening, and withdrawing biogas via the at least one draw opening. The stirrer devices are driven by a drive device in at least two groups. Each group includes at least one stirrer device. The stirrer devices of a first group are driven in at least one first time interval. Stirrer devices of a second group are stationary in the first time interval.

### CROSS-REFERENCE TO RELATED APPLICATIONS

This application is a continuation application of international patent application PCT/EP2022/072479, filed Aug. 10, 2022, designating the United States, and the entire content of which is incorporated herein by reference.

### BACKGROUND

US 2010/0062482 has disclosed a method for operating a fermentation device, the fermentation device having an elongate container and multiple stirring devices which are arranged in the container. The stirring devices serve here both for moving and mixing the substrate in the container and for transporting sediment at the base of the container.

### SUMMARY

It is an object of the disclosure to specify a method for operating a fermentation device by way of which energy-saving operation of the fermentation device is possible.

The object is, for example, achieved by a method for operating a fermentation device, wherein the fermentation device includes an elongate container, wherein the container defines at least one introduction opening at a first end face of the elongate container, at least one discharge opening at a second end face of the container disposed opposite to the first end face, and at least one extraction opening for biogas. The fermentation device further includes a plurality of stirring devices and at least one drive device for the stirring devices, each of the stirring devices has at least one stirrer shaft arranged transversely to a longitudinal axis of the container and configured to be driven in rotation about a central axis of the stirrer shaft by a drive device, the stirring device having at least one stirrer blade which is fixed to the stirrer shaft and which projects outward. The method includes: introducing substrate containing organic material via the at least one introduction opening; moving and mixing the substrate in the container via the stirring devices that are driven in rotation; discharging treated material via the at least one discharge opening; removing biogas via the at least one extraction opening; and, wherein the plurality of stirring devices are driven in at least two groups by the drive device including a first group of stirring devices and a second group of stirring devices, wherein each of the at least two groups includes at least one of the plurality of stirring devices, wherein the first group of stirring devices are driven in at least one first time interval, and wherein the second group of stirring devices are at a standstill in the first time interval.

It is provided that the stirring devices are driven by the drive device in at least two groups. Each group of stirring devices includes here at least one stirring device. In order to allow energy-saving operation, it is provided that the stirring devices of a first group are driven in at least one first time interval and the stirring devices of a second group are at a standstill in the first time interval. Accordingly, not all the stirring devices of the fermentation device are driven simultaneously in the first time interval. As a result, the energy outlay for operating the stirring devices can be lowered significantly. It has been found that, during the standstill of the stirring devices of the second group in the first time interval, there are surprisingly no resulting adverse effects on sediment transport or destruction of the floating cover. There is no consequent negative influence on the maintenance of a plug-flow characteristic in the container and the vertical mixing, particularly the degassing of the substrate, either.

Advantageously, over at least 50%, in particular over at least 80%, of the operating duration of the fermentation device, not all the stirring devices are driven. Particularly preferably, at no time are all the stirring devices driven simultaneously. As a result, the energy consumption can be lowered significantly in comparison with an operation during which all the stirring devices are driven simultaneously.

Preferably, the stirring devices of the first group are driven in a second direction of rotation, which is opposite to the first direction of rotation, in at least one second time interval and the stirring devices of the second group are at a standstill in the second time interval. The driving of the stirring devices of the first group in the second direction of rotation results in a return transport of substrate within the container being realized. Preferably, the time intervals are configured in such a way that external recirculation of treated material and a device for preconditioning the substrate or a device for upstream mixing of fresh substrate with treated material can be dispensed with completely during normal operation. Normal operation is any operation beside start-up and troubleshooting. During start-up or troubleshooting, provision may be made for material treated in the container to be recirculated. It has also been found that the driving of the stirring devices of the first group in the second direction of rotation surprisingly results in the vertical mixing and the degassing of the substrate being able to be positively influenced. It is also possible for the plug-flow characteristic in the container to be maintained or even improved. A negative influence on sediment transport and on the destruction of floating cover is also not observed when the stirring devices of the first group are driven in the second direction of rotation.

The first time interval is in particular greater than or equal to the second time interval. The fact that the first time interval is at least as great as the second time interval means that sufficient sediment transport at the base of the container can be ensured.

A preferred arrangement is obtained if the stirring devices are driven in such a way that stirring devices of a group are not successive in the direction of the longitudinal axis. The first direction of rotation is preferably oriented in such a way that the stirrer blades, at the base of the container, move in the direction from the first end face to the second end face of the container.

In order to ensure that the substrate supplied to the container is mixed in well, it is provided that, during the introduction of substrate, at least the stirring device which adjoins the introduction opening is driven in the first direction of rotation. Good removal of the treated material can be ensured in a simple manner if, during the removal of treated material, at least the stirring device which adjoins the at least one discharge opening is driven in the first direction of rotation.

The energy requirement of the fermentation device can be further reduced in a simple manner in that, in a third time interval, all the stirring devices of the first group and of the second group, in particular all the stirring devices of the fermentation device, are at a standstill.

Each group of stirring devices includes at least one stirring device. Accordingly, in the first time interval and in particular also in the second time interval, at least one stirring device is at a standstill. Preferably, each group includes two stirring devices to six stirring devices.

Preferably, every time interval in which the stirring devices of a group are driven in rotation is followed by a time interval in which the stirring devices of the group are at a standstill. The time interval over which the stirring devices of one group are driven advantageously corresponds to an integer multiple of half-revolutions of the stirring devices. Consequently, it can be ensured in a simple manner that the stirring devices are situated at a standstill in a defined position in which they cannot collide with adjacent stirring devices. The integral multiple is in this case preferably from 2 to 10. However, other time intervals for the driving of the stirring devices may also be advantageous. The time interval over which the stirring devices of one group are at a standstill advantageously corresponds to a half-revolution or an integer multiple of half-revolutions of the stirring devices. It is preferably provided that the integer multiple is from 2 to 10.

The rotational speed of the stirring devices is advantageously set according to a predefined nominal rotational speed via a frequency converter. The rotational speed is preferably set in the range of 80% to 100% of the nominal rotational speed. The nominal rotational speed is advantageously 0.5 rpm to 2 rpm, in particular approximately 1 rpm. However, other rotational speeds and/or other controls for the rotational speed may also be advantageous.

For the introduction of substrate, it is advantageously provided that the substrate is introduced in successive time periods. For each time period, the introduction duration or the substrate quantity is advantageously predefined. Preferably, the substrate is not introduced continuously. The introduction duration of the substrate is preferably 10% to 60% of the time period. The time period may for example be 0.5 h to 2 h, preferably 0.75 h to 1.5 h, particularly preferably approximately 1 h.

Advantageously, the substrate is moved in the fermentation device from the at least one introduction opening to the at least one discharge opening exclusively by way of feeding and removal and via the stirring devices. Additional devices for transporting substrate, in particular sediments of the substrate, are advantageously not provided. This results in a simple construction of the fermentation device being achieved.

The fermentation device is advantageously a plug-flow fermenter.

To ensure good transport in particular of sediments at the base of the container, it is advantageously provided that each stirrer blade has a maximum extent in relation to the central axis of the stirring device, wherein the axis spacing of the central axes of at least two stirring devices following one another in the direction of the longitudinal axis of the container is preferably less than or equal to the sum of the maximum radial extents of the two stirring devices. The areas passed through by the stirrer blades of stirring devices following one another in the direction of the longitudinal axis of the container overlap. The sediments are transported in the manner of shifting-sand dunes. At the base of the container, a leading stirrer blade forms a dune by accumulation, which dune is stripped down by the following stirrer blade on that side of the dune which faces toward the discharge opening. In this way, sediment transport may be realized at the base of the container without additional devices in a simple manner.

### BRIEF DESCRIPTION OF DRAWINGS

The invention will now be described with reference to the drawings wherein:

FIG. 1 shows a sectional illustration through a fermentation device;

FIG. 1A shows a schematic illustration of an alternative arrangement of adjacent stirring devices;

FIG. 2 shows a schematic cross section through the container of the fermentation device from FIG. 1; and,

FIGS. 3 and 4 show diagrams for the driving of the stirring devices in the first or the second direction of rotation over time.

### DETAILED DESCRIPTION

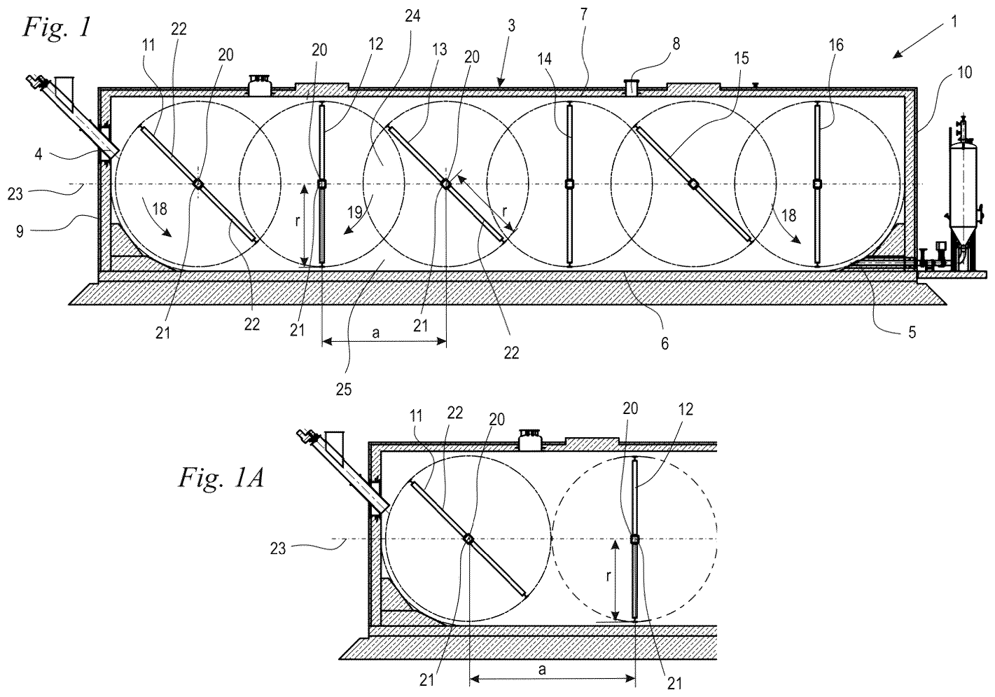

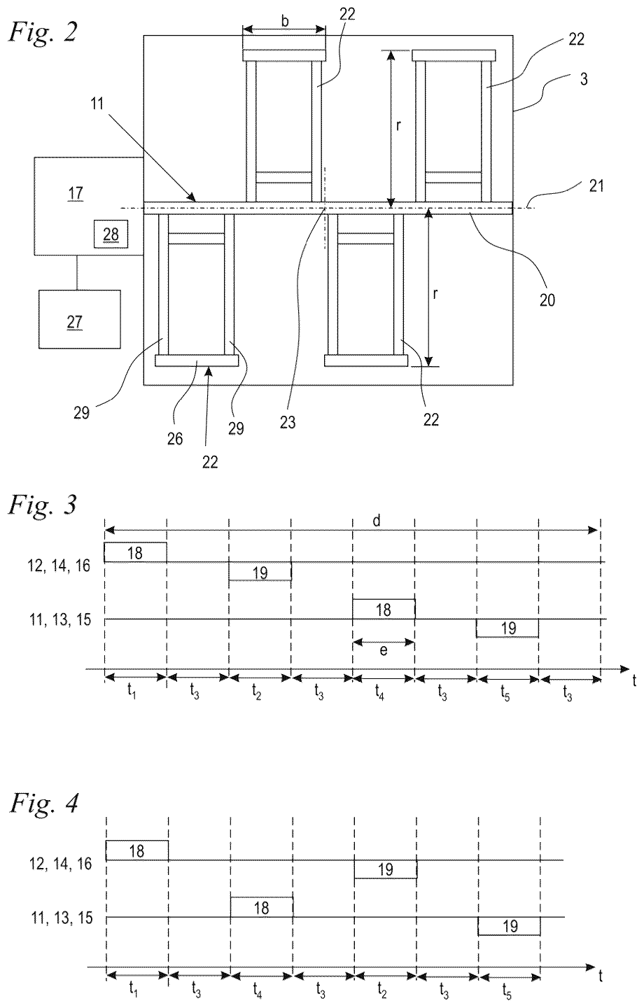

FIG. 1 schematically shows a fermentation device 1 in a longitudinal section. The fermentation device includes a container 3 which has a first end face 9 and second end face 10. The container 3 has a longitudinal axis 23 which extends through the first end face 9 and the second end face 10. The container 3 is arranged horizontally, and the longitudinal axis 23 extends horizontally in the embodiment. The container 3 has an introduction opening 4 for substrate at the first end face 9 and a discharge opening 5 for treated material at the second end face 10. At a container top 7, situated at the top, of the container 3, provision is made of an extraction opening 8 for biogas in the embodiment. It is also possible for provision to be made of multiple introduction openings 4, discharge openings 5 and/or extraction openings 8. Preferably, provision is made of introduction openings 4 only at the first end face 9 and of discharge openings for treated material exclusively at the second end face 10. Alternatively, provision may additionally be made of further introduction openings 4 between the end faces 9 and 10. That side of the container 3 which is situated at the bottom forms a container base 6. Preferably, the container base 6 and the container top 7 are planar and oriented parallel to one another, as shown in FIG. 2.

The fermentation device 1 is configured as a so-called plug-flow fermenter. In such a plug-flow fermenter, the substrate moves horizontally in the container 3. The substrate is transported by way of feeding and removal and also via stirring devices 11, 12, 13, 14, 15, 16, the configuration of which will be explained in more detail below on the basis of the first stirring device 11. The further stirring devices 12 to 16 may be configured in a corresponding manner. Further devices for moving substrate in the container 3 are advantageously not provided.

The first stirring device 11 includes a stirrer shaft 20 which is able to be driven in rotation about a central axis 21. The central axis 21 is oriented transversely, preferably perpendicularly, to the longitudinal axis 23. At least one stirrer blade 22 extends outward from the stirrer shaft 20. In the embodiment, provision is made of at least two stirrer blades 22 extending on opposite sides of the stirrer shaft 20. The stirrer blades 22 have a maximum radial extent r in relation to the central axis 21 of the associated stirring device. In the embodiment, the maximum radial extent r is the same for all the stirrer blades 22 of all the stirring devices 11 to 16. Different maximum radial extents r for stirrer blades 22 of a stirring device 11 to 16 or for stirrer blades 22 of different stirring devices 11 to 16 may also be advantageous, however. The central axes 21 of two stirring devices 11 to 16 following one another in the direction of the longitudinal axis 23 of the container 3 have an axis spacing a. The axis spacing a between the central axes 21 of the stirrer shafts 20 of the second stirring device 12 and of the third stirring device 13 is illustrated by way of example in FIG. 1. In the embodiment, the central axes 21 of adjacent stirring devices 11 to 16 have the same axis spacing a in all cases.

In the embodiment, the axis spacing a is less than the sum of the maximum radial extents r of the stirrer blades 22 of adjacent stirring devices 11 to 16. The fact that the axis spacing a is less than the sum of the maximum radial extents r of the respectively adjacent stirring devices 11 to 16 means that adjacent stirring devices 11 to 16 form an overlap region 24, which is indicated for the second stirring device 12 and the third stirring device 13 in FIG. 1. The overlap region 24 is passed through both by a stirrer blade 22 of the stirring device 12 and by a stirrer blade 22 of the stirring device 13.

During operation, a sediment accumulation 25 more or less in the shape of a dune accumulates on the container base 6 below the overlap region 24. The second stirring device 12 piles the sediments on the sediment accumulation 25. On that side of the sediment accumulation 25 which is closer to the end face 10, sediment is carried along, and transported away in the direction of the discharge opening 5, by the following third stirring device 13. Preferably, between adjacent stirring devices 11 to 16, provision is made in all cases of corresponding overlap regions 24. Owing to the overlapping of adjacent stirring devices 11 to 16, it is advantageously possible to dispense with an additional device for transporting sediments.

An alternative arrangement of two adjacent stirring devices is illustrated schematically in FIG. 1A for the stirring devices 11 and 12. In this embodiment, the axis spacing a is equal to the sum of the maximum radial extents r of the stirrer blades 22 of adjacent stirring devices 11 to 16. In the case of this arrangement, too, the sediments can be transported in the manner described above.

During the operation of the fermentation device 1, substrate containing organic material is supplied into the container 3 via the introduction opening 4. During the introduction of substrate, the first stirring device 11, which adjoins the first end face 9, rotates in a first direction of rotation 18. The direction of rotation 18 is directed in such a way that, at the container base 6, the stirrer blades 22 move from the first end face 9 in the direction of the second end face 10. At the container top 7, the stirrer blades 22 move in the opposite direction, that is, from the second end face 10 in the direction of the first end face 9. In the embodiment, the central axes 21 of the stirrer shafts 20 are oriented horizontally and perpendicularly to the longitudinal axis 23. All the central axes 21 extend parallel to one another. Due to the driving of the first stirring device 11 in the first direction of rotation 18, substrate supplied via the introduction opening 4 is transported onward rapidly. This avoids overloading of the fermentation device 1 in the feeding region. Here, during normal operation, the substrate is supplied directly via the introduction opening 4 without conditioning of the substrate ahead of the fermentation device 1 or mixing with material that has already been treated taking place.

The fermentation device 1 illustrated in FIG. 1 has no external recirculation means. Treated material that has been discharged from the container 3 via the discharge opening 5 is accordingly not conducted back to the introduction opening or to a preconditioning device or to a forced mixer again, in order to be added to the fresh substrate, but is discharged completely. Recirculation of treated substrate takes place in the container 3 itself. In order to make this recirculation possible, provision is made for at least one of the stirring devices 11 to 16 to be driven intermittently in a second direction of rotation 19, which is directed counter to the first direction of rotation 18. The direction of rotation 19 is indicated for the second stirring device 12 in FIG. 1.

The driving of the stirring devices 11 to 16 in the first direction of rotation 18 and the second direction of rotation 19 is described in more detail below. The sixth stirring device 16, which adjoins the discharge opening 5 and the second end face 10, is preferably driven in the first direction of rotation 18 during the extraction of treated material from the container 3, so that the stirrer blades 22 move in the direction of the second end face 10 adjacent to the container base 6 and thus realize the sediment transport at the container base 6 to the discharge opening 5.

FIG. 2 schematically shows a section through the first stirring device 11. The further stirring devices 12 to 16 are preferably of identical form. In the embodiment, provision is made of a drive device 17, for example a drive motor with or without a transmission unit, for each stirring device 11 to 16. It may also be provided that a drive device 17 drives a multiple number of the stirring devices 11 to 16 or all the stirring devices 11 to 16 via suitable transmission devices, such as belt drives or the like. The fermentation device 1 includes a control device 27 which suitably controls the at least one drive device 17. Here, it is provided that none of the drive devices 17 runs without interruption, but rather that the drive devices 17 are in operation only intermittently. This allows the energy requirement of the fermentation device 1 to be reduced in a simple manner. The drive device 17 includes a frequency converter 28, via which the rotational speed of the stirrer shaft 20 is settable in a simple manner.

As also shown in FIG. 2, four stirrer blades 22 are arranged on a common stirrer shaft 20 in the embodiment. The stirrer blades 22 are respectively formed by two outwardly projecting arms 29 which, at their radially outer end, carry a horizontally extending stirring bar 26. The stirring bar 26 extends over the entire width b of each stirrer blade 22. Some other configuration of the stirrer blades 22 may also be advantageous.

As shown in FIG. 2, oppositely situated stirrer blades 22 are arranged offset from one another in the direction of the central axis 21. Thus, each stirrer blade 22 moves about the central axis 21 in its own disk-shaped region. With regard to the sectional illustration illustrated in FIG. 2, the stirrer blades 22 of a stirring device 11 to 16 do not overlap. Stirrer blades 22 following one another in the direction of the central axis 21 are advantageously arranged on opposite sides of the central axis 21.

The stirring devices 11 to 16 are advantageously driven in at least two groups. Preferably, each group of stirring devices 11 to 16 includes at least one, preferably at least two, stirring devices 11 to 16. FIGS. 3 and 4 show, by way of example, two possible types of control of the stirring devices 11 to 16, in the case of which the stirring devices are divided into two groups. In the embodiment, a first group includes the stirring devices 12, 14 and 16 and a second group includes the stirring devices 11, 13 and 15.

In a first time interval t₁, for the temporal sequence, illustrated in FIG. 3, for the driving of the stirring devices 11 to 16, provision is made for the stirring devices 12, 14 and 16 to be driven in the first direction of rotation 18. In a later second time interval t₂, the stirring devices 12, 14 and 16, which form the first group, are driven in the second, opposite, direction of rotation 19. Between the first time interval t₁ and the second time interval t₂, there is a third time interval t₃, during which the stirring devices 12, 14 and 16 of the first group are at a standstill. The stirring devices 11, 13 and 15 of the second group are at a standstill during the first time interval t₁, the second time interval t₂ and the third time interval t₃. The second time interval t₂ is followed by a further third time interval t₃, during which again all the stirring devices 11 to 16 of both groups are at a standstill. In a fourth time interval t₄ that follows, the stirring devices 11, 13, and 15 of the first group are driven in the first direction of rotation 18. In t fourth time interval t₄, substrate may be supplied into the container 1 via the input opening 4. In the first time interval t₁, treated material may be extracted from the discharge opening 5 via the discharge opening 5. The fourth time interval t₄ is followed again by a third time interval t₃, during which all the stirring devices 11 to 16 are at a standstill. In the fifth time interval t₅ that follows, the stirring devices 11, 13 and 15 of the second group are driven in a second direction of rotation 19.

In an alternative process sequence, the supply of substrate and the extraction of treated material are independent of which of the groups of stirring devices 11 to 16 is being driven or is at a standstill. For example, it is possible for substrate to be supplied over a specific length of time in any time interval t₁, t₂, t₄, t₅. Advantageously, the first stirring device 11 is driven in the first direction of rotation 18 independently of the further stirring devices 13 and 15 of the group while the substrate supply is taking place. Correspondingly, the sixth stirring device 16 is advantageously driven in the first direction of rotation 18 independently of the further stirring devices 12 and 14 of the group while treated material is being extracted.

FIG. 4 indicates alternative driving of the stirring devices 11 to 16. Firstly, the stirring devices 12, 14 and 16 of the first group are driven in the first direction of rotation 18, specifically in the first time interval t₁. This is followed by a third time interval t₃, during which all the stirring devices 11 to 16 are at a standstill. In a fourth time interval t₄ that follows, the stirring devices 11, 13 and 15 of the second group are driven in the first direction of rotation 18. This is followed again by a third time interval t₃, during which none of the stirring devices 11 to 16 is driven. Following this, the stirring devices 12, 14 and 16 of the first group are driven in the second direction of rotation 19 in a second time interval t₂. After the following third time interval t₃, during which all the stirring devices 11 to 16 are at a standstill, the stirring devices 11, 13 and 15 of the second group are driven in the second direction of rotation 19 in the fifth time interval t₅. While the stirring devices of one group are being driven, the stirring devices of the other group are advantageously at a standstill. Overall, this results in a relatively short operating duration of each stirring device 11 to 16, whereby the energy requirement of the fermentation device 1 can be significantly lowered.

The first time interval t₁, during which the stirring devices 12, 14 and 16 are driven in the first direction of rotation 18, is preferably greater than or equal to the second time interval t₂, in which the stirring devices 12, 14 and 16 are driven in the opposite direction of rotation 19. As shown in FIG. 1, stirring devices following one another are assigned to different groups.

The time intervals t₁, t₂, t₄ and t₅, during which the stirring devices of one group are driven, advantageously correspond to integral multiples of half-revolutions of the stirring devices 11 to 16. The integral multiples are advantageously from 2 to 10. It is also the case that the third time interval t₃, during which the stirring devices 11 to 16 of both groups are at a standstill, corresponds preferably to a half-revolution or to an integral multiple of half-revolutions of the stirring devices 11 to 16. The integral multiple is in this case advantageously from 2 to 6. The rotational speed of the stirring devices 11 to 16 can advantageously be set according to the substrate introduced. The frequency converter 27 illustrated in FIG. 2 serves for this purpose. The rotational speed is advantageously set in the range of 80% to 100% of the nominal rotational speed. The nominal rotational speed is preferably 0.5 rpm to 2 rpm, particularly preferably approximately 1 rpm. Accordingly, the stirrer blades 22 move relatively slowly through the substrate in the container 3.

The supply of the substrate via the introduction opening 4 (FIG. 1) advantageously takes place virtually continuously in successive time periods d. The supply advantageously takes place once during each time period d. The introduction duration e or the substrate quantity for each time period d is advantageously predefined. FIG. 3 illustrates, by way of example, a time period d which encompasses the driving of each stirring device 11 to 16 in each direction of rotation exactly once. Substrate is introduced over an introduction duration e which is 10% to 60% of the time period d. In the embodiment, the introduction duration e corresponds to the fourth time interval t₄, in which the first stirring device 11 is driven in the first direction of rotation 18. The time period d is advantageously 0.5 h to 2 h, in particular 0.75 h to 1.5 h, preferably approximately 1 h. The stirring devices 11 to 16 move the substrate in the container 3 and mix the substrate. Some other choice of the time period d and the introduction duration e may also be advantageous. Preferably, the time period d is significantly greater than the time intervals t₁ to t₅. The time intervals t₁, t₂, t₄ and t₅, during which the stirring devices of one group are driven, are advantageously an integral multiple of half-revolutions of the stirring devices 11 to 16. The integral multiple is in this case preferably from 2 to 10. The time intervals t₁ to t₅, during which the stirring devices of one group are at a standstill, are advantageously an integral multiple of half-revolutions of the stirring devices 11 to 16. The integral multiple is in this case preferably from 2 to 10. The rotational speed of the stirring devices 11 to 16 is advantageously 80% to 100% of a nominal rotational speed. The nominal rotational speed is advantageously 0.5 rpm to 2 rpm, preferably 1 rpm. The time period d is advantageously significantly greater than the time intervals t₁ to t₅.

It may be provided that the time intervals t₁ to t₅ are of equal length. Time intervals t₁ to t₅ of different lengths may also be advantageous. Advantageously, time intervals t₄ and t₅, during which the stirring devices of one group are driven in the second direction of rotation 19, are not greater than the time intervals t₁ and t₂, during which the stirring devices of one group are driven in the first direction of rotation 18.

Through suitable selection of the time intervals t₁ to t₅ and suitable division of the stirring devices 11 to 16 into groups of in each case at least one, preferably of two to six, stirring devices and owing to the at least intermittent driving of at least one stirring device 11 to 16 in the second direction of rotation 19, recirculation of substrate can take place within the container 3. External recirculation of treated material and preconditioning are not required during normal operation. This makes possible a fermentation device 1 of simple construction that has a low energy requirement during operation. The stirring devices 11 to 16 bring about vertical mixing of the substrate, destruction of the floating cover, and distribution and transport of sediments of the substrate. The supply of the substrate into the container 4 takes place virtually continuously. The stirring devices 11 to 16 are in an intermittent-operation mode and are controlled only according to the program for driving the stirring devices 11 to 16 that is stored in the control device 27. The stirring devices 11 to 16 are advantageously operational only for a short time in each case. It is possible here for the first stirring device 11 and the last stirring device 16 to be driven additionally and independently of the further stirring devices of the respective group during introduction of substrate and removal of treated material, and to thus have longer operating times than the other stirring devices.

Due to the at least intermittent movement of at least one stirring device in the second direction of rotation 19, it is possible for the substrate to be recirculated within the container 3 and to be mixed with the supplied material in the container 3. In this way, desired dry-matter contents, in particular in the first stirring device 11, and desired dilution for reducing the viscosity are settable. Advantageously, the dry-matter content of the substrate supplied into the container 2 is less than 45% by weight, in particular 30% by weight to 45% by weight. The fermentation device 1 is advantageously a fermentation device for continuous dry fermentation. The at least intermittent driving of at least one stirring device 11 to 16 in the second direction of rotation 19 makes it possible to maintain the plug-flow characteristic in the container 3 and to realize vertical mixing and degassing of the substrate. The sediment transport at the container base 6 and the destruction of the floating cover in the container 3 remain ensured.

The substrate supplied to the fermentation device 1 advantageously has a dry-substance content of at least 20% by weight.

The substrate supplied to the fermentation device 1 includes in particular different domestic or commercial organic wastes, such as for example separately collected biowastes, organic-substance-containing fine fractions from mixed household waste, green wastes or separately collected food wastes from households or restaurants. Alternatively or additionally, the substrate supplied to the fermentation device 1 includes wastes with seasonally or constantly varying properties or compositions and/or with relatively large proportions of impurities, such as for example non-fermentable hard or inert materials such as stones, glass, ceramic, grit or the like. The substrate supplied to the fermentation device 1 includes in particular also relatively highly viscous, structure-rich or else fibrous substrates from agriculture, landscape conservation, commerce and industry, such as for example straw, grass, silages or other cellulose-containing material flows, for example from the paper industry, and/or dewatered sewage sludges.

It is understood that the foregoing description is that of the preferred embodiments of the invention and that various changes and modifications may be made thereto without departing from the spirit and scope of the invention as defined in the appended claims.

1. A method for operating a fermentation device, wherein the fermentation device includes an elongate container, wherein the container defines at least one introduction opening at a first end face of the elongate container, at least one discharge opening at a second end face of the container disposed opposite to the first end face, and at least one extraction opening for biogas, the fermentation device further including a plurality of stirring devices and at least one drive device for the stirring devices, each of the stirring devices has at least one stirrer shaft arranged transversely to a longitudinal axis of the container and configured to be driven in rotation about a central axis of the stirrer shaft by a drive device, the stirring device having at least one stirrer blade which is fixed to the stirrer shaft and which projects outward, wherein the method comprises:
introducing substrate containing organic material via the at least one introduction opening;
moving and mixing the substrate in the container via the stirring devices that are driven in rotation;
discharging treated material via the at least one discharge opening;
removing biogas via the at least one extraction opening; and,
wherein the plurality of stirring devices are driven in at least two groups by the drive device including a first group of stirring devices and a second group of stirring devices, wherein each of the at least two groups includes at least one of the plurality of stirring devices, wherein the first group of stirring devices are driven in at least one first time interval, and wherein the second group of stirring devices are at a standstill in the first time interval.

2. The method of claim 1, wherein the first group of stirring devices are driven in a second direction of rotation, which is opposite to a first direction of rotation, in at least one second time interval; and, the second group of stirring devices are at a standstill in the second time interval.

3. The method of claim 2, wherein the first time interval is greater than or equal to the second time interval.

4. The method of claim 1, wherein the plurality of stirring devices are driven such that individual ones of the first group of stirring devices and of the second group of stirring devices are not successive in the direction of the longitudinal axis.

5. The method of claim 1, wherein, during the driving in a first direction of rotation, the stirrer blades, at the base of the elongate container, move in a direction from the first end face to the second end face of the elongate container.

6. The method of claim 1, wherein, during said introducing the substrate, at least one stirring device of the plurality of stirring devices which adjoins the introduction opening is driven in a first direction of rotation.

7. The method of claim 1, wherein, during said removal of treated material, at least stirring device of the plurality of stirring devices which adjoins the at least one discharge opening is driven in a first direction of rotation.

8. The method of claim 1, wherein, in a third time interval, all of the first group of stirring devices and all of the second group of stirring devices are at a standstill.

9. The method of claim 1, wherein, in a third time interval, all the plurality of stirring devices of the fermentation device are at a standstill.

10. The method of claim 1, wherein the first group of stirring devices and the second group of stirring devices each include 2 to 6 stirring devices.

11. The method of claim 1, wherein every time interval in which the first group of stirring devices are driven in rotation is followed by a further time interval in which the first group of stirring devices are at a standstill; and, every time interval in which the second group of stirring devices are driven in rotation is followed by a further time interval in which the second group of stirring devices are at a standstill.

12. The method of claim 1, wherein the substrate is introduced in successive time periods, wherein, for each time period, an introduction duration or a substrate quantity is predefined.

13. The method of claim 12, wherein the introduction duration is 10% to 60% of the time period.

14. The method of claim 12, wherein the time period is 0.5 hours to 2 hours.

15. The method of claim 12, wherein the time period is 1 hour.

16. The method of claim 1, wherein the substrate is moved in the fermentation device from the at least one introduction opening to the at least one discharge opening exclusively by way of feeding and removal and via the plurality of stirring devices.

17. The method of claim 1, wherein each stirrer blade has a maximum radial extent in relation to the central axis of the stirring device; an axis spacing of the central axes of at least two of the plurality of stirring devices following one another in the direction of the longitudinal axis of the elongate container is less than or equal to a sum of a maximum radial extents of the two of the plurality of stirring devices.
